(19) United States
(12) Patent Application Publication
(10) Pub. No.: US 2001/0055760 A1
(43) Pub. Date: Dec. 27, 2001

(54) NUCLEIC ACID ARRAYS

(76) Inventor: ALEX CHENCHIK, PALO ALTO, CA (US)

Correspondence Address: BRET FIELD, BOZICEVIC FIELD & FRANCIS LLP, 200 MIDDLEFIELD ROAD, SUITE 200, MENLO PARK, CA, 94025, US

(21) Appl. No.: 09/417,268

(22) Filed: Oct. 13, 1999

Related U.S. Application Data
(60) Provisional application No. 60/104,179, filed on Oct. 13, 1998.

(51) Int. Cl. C12Q 1/68; G01N 1/00; C12M 3/00; C12M 1/34; G01N 21/76; G01N 21/00

(52) U.S. Cl. 435/6; 435/287.2; 435/288.3; 422/50; 422/52; 422/58

(57) ABSTRACT

Arrays of oligonucleotide spots, as well as methods for their production and use, are provided. The subject arrays have at least one pattern of probe oligonucleotide spots stably associated with the surface of a solid support. A plurality of different target nucleic acids are represented in the pattern, where each target nucleic acid may correspond to one probe oligonucleotide spot or a plurality of different probe oligonucleotide spots. In one type of preferred embodiment, all of the oligonucleotide spots correspond to the same type of target nucleic acid, i.e. all of the corresponding target nucleic acids are the same type of gene. Each probe oligonucleotide spot is made up of a plurality of unique oligonucleotides that are capable of hybridizing to different regions of the corresponding target nucleic acid. The subject arrays find use in hybridization assays, particularly in assays for the identification of differential gene expression patterns among cells.

### TECHNICAL FIELD

The field of this invention is biopolymeric arrays.

### BACKGROUND OF THE INVENTION

“Biochips” or arrays of binding agents, such as oligonucleotides and peptides, have become an increasingly important tool in the biotechnology industry and related fields. These binding agent arrays, in which a plurality of binding agents are deposited onto a solid support surface in the form of an array or pattern, find use in a variety of applications, including drug screening, nucleic acid sequencing, mutation analysis, and the like. One important use of biochips is in the analysis of differential gene expression, where the expression of genes in different cells, normally a cell of interest and a control, is compared and any discrepancies in expression are identified. In such assays, the presence of discrepancies indicates a difference in the classes of genes expressed in the cells being compared.

In methods of differential gene expression, arrays find use by serving as a substrate to which is bound nucleic acid “probe” fragments. One then obtains “targets” from analogous cells, tissues or organs of a healthy and diseased organism. The targets are then hybridized to the immobilized set of nucleic acid “probe” fragments. Differences between the resultant hybridization patterns are then detected and related to differences in gene expression in the two sources.

A variety of different array technologies have been developed in order to meet the growing need of the biotechnology industry, as evidenced by the extensive number of patents and references listed in the relevant literature section below.

Despite the wide variety of array technologies currently in preparation or available on the market, there is a continued need to improve the performance of arrays and identify new array devices to meet the needs of specific applications. Of particular interest would be the development of an array capable of providing high throughput analysis of differential gene expression.

Relevant Literature

Patents and patent applications describing arrays of biopolymeric compounds and methods for their fabrication include: U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,895; 5,624,711; 5,639,603; 5,658,734; WO 93/17126; WO 95/11995; WO 95/35505; EP 742 287; and EP 799 897.

Patents and patent application describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; WO 95/21265; WO 96/31622; WO 97/10365; WO 97/27317; EP 373 203; and EP 785 280.

Other references of interest include: Atlas Human cDNA Expression Array I (April 1997) CLONTECHniques XII: 4-7; Lockhart et al., Nature Biotechnology (1996) 14: 1675-1680; Shena et al., Science (1995) 270: 467-470; Schena et al., Proc. Nat'l Acad. Sci. USA (1996) 93:10614-10619; Shalon et al., Genome Res. (1996) 6: 639-645; Milosavljevic et al., Genome Res. (1996) 6:132-141; Nguyen et al., Genomics (1995) 29: 207-216; Pietu et al., Genome Res. (1996) 6: 492-503; Zhao et al., Gene (1995) 166:207-213; Chalifour et al., Anal. Biochem. (1994) 216:299-304; Heller et al., Proc. Nat'l Acad. Sci. USA (1997) 94: 2150-2155; O'Meara et al., Analytical Biochemistry (1988) 255: 195-203; and Schena, M., BioAssays (1996) 18: 427-431.

### SUMMARY OF THE INVENTION

Arrays of oligonucleotide spots stably associated with the surface of a solid support, as well as methods for their preparation and use in hybridization assays, are provided. The oligonucleotide spots of the subject arrays comprise an oligonucleotide composition of a plurality of unique oligonucleotides that serve as probes and are capable of hybridizing to different regions of a corresponding target nucleic acid. A plurality of target nucleic acids are represented on the array, where each target may be represented by a single probe spot on the array or a plurality of different probe spots on the array. In a preferred embodiment, all of the target nucleic acids represented on the array are of the same type, i.e. all of the probe spots on the array correspond to the same type of gene. The subject arrays find particular use in differential gene expression analysis.

### DEFINITIONS

The term “nucleic acid” as used herein means a polymer composed of nucleotides, e.g. deoxyribonucleotides or ribonucleotides.

The terms “ribonucleic acid” and “RNA” as used herein means a polymer composed of ribonucleotides.

The terms “deoxyribonucleic acid” and “DNA” as used herein means a polymer composed of deoxyribonucleotides.

The term “oligonucleotide” as used herein denotes single stranded nucleotide multimers of from about 10 to 150 nucleotides in length.

The term “polynucleotide” as used herein refers to single or double stranded polymer composed of nucleotide monomers of greater than about 150 nucleotides in length up to about 3000 nucleotides in length.

The term “array type” refers to the type of gene represented on the array by the unique oligonucleotides, where the type of gene that is represented on the array is dependent on the intended purpose of the array, e.g. to monitor expression of key human genes, to monitor expression of known oncogenes, to measure toxicity of different drug compounds by monitoring expression of stress response and other related genes, etc., i.e. the use for which the array is designed. As such, all of the unique oligonucleotides on a given array correspond to the same type or category or group of genes. Genes are considered to be of the same type if they share some common linking characteristics, such as: species of origin, e.g. human, mouse, rat, viruses, etc.; organ, tissue or cell type of origin, e.g. muscle, endocrine glands, blood, neural, dermal, etc.; disease state, e.g. cancer; metabolic disorder related genes, functions, e.g. protein kinases, tumor suppressors, G-protein coupled receptors, and the like, participation in the same normal biological process, e.g. apoptosis, signal transduction, cell cycle regulation, proliferation, differentiation, aging, etc.; and the like. For example, one array type that is provided below is a “cancer array” in which each of the “unique” oligonucleotide probes correspond to a gene associated with a cancer disease state. Likewise, a “human array” may be an array of oligonucleotides corresponding to unique tightly regulated human genes. Similarly, an “apoptosis array” may be an array type in which the oligonucleotides correspond to unique genes associated with apoptosis. Other representative types of arrays include: mouse array, human stress/toxicology array, oncogene and tumor suppressor array, cell-cell interaction array, cytokine and cytokine receptor array, rat array, rat stress/toxicology array, hematology array, mouse stress/toxicology array, neuroarray, drug target array, cardiovascular array, aging array, differentiation array, signal transduction pathways array, fat metabolism array, inflammation array, viral-host interaction array, and the like.

The “unique” oligonucleotide sequences associated with each type of array of the present invention are sequences which are distinctive or different with respect to every other oligonucleotide sequence on the array. For example, in a cancer array, each unique oligonucleotide has a sequence that is not homologous to any other known cancer associated sequence. Moreover, each oligonucleotide sequence on the array is statistically chosen to ensure that the probability of homology to any sequence of that type is very low. Morever, in each array embodiment, all sequences are statistically chosen to insure that probability of homology to any other sequence associated with cancer or of human origin is very low. An important feature of the individual oligonucleotide probe compositions of the subject arrays is that they are only a fragment of the entire cDNA of the gene to which they correspond. In other words, for each gene represented on the array, the entire cDNA sequence of the gene is not represented on the array. Instead, the sequence of only a portion or fragment of the entire cDNA is represented on the array by each unique oligonucleotide.

The term “oligonucleotide probe composition” refers to the nucleic acid composition that makes up each of the spots on the array that correspond to a target nucleic acid. Thus, oligonucleotide probe compositions are nucleic acid compositions of unique oligonucleotides. The oligonucleotide compositions are made up of a plurality of unique oligonucleotides that are capable of hybridizing to different (either over-lapping or separate) regions, i.e. stretches of nucleotides or domains, of the target nucleic acid to which they correspond.

The term “target nucleic acid” means a nucleic acid for which there is one or more corresponding oligonucleotide probe compositions, i.e. probe oligonucleotide spots, present on the array. The target nucleic acid may be represented by one or more different oligonucleotide probe compositions on the array. The target nucleic acid is a nucleic acid of interest in a sample being tested with the array, where by “of interest” is meant that the presence or absence of target in the sample provides useful information, e.g. unique and defining characteristics, about the genetic profile of the cell(s) from which the sample is prepared. As such, target nucleic acids are not housekeeping genes or other types of genes which are present in a number of diverse cell types and therefore the presence or absence of which does not provide characterizing information about a particular cell's genetic profile.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid.

The terms “mismatch control” or “mismatch probe” refer to probes whose sequence is deliberately selected not to be perfectly complementary to a particular target sequence. For each mismatch (MM) control in an array there typically exists a corresponding perfect match (PM) probe that is perfectly complementary to the same particular target sequence. The mismatch may comprise one or more bases. While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

### DESCRIPTION OF THE SPECIFIC EMBODIMENTS

Arrays of oligonucleotide spots and methods for their preparation are provided. In the subject arrays, a plurality of oligonucleotide spots is stably associated with the surface of a solid support. The oligonucleotide probe composition of each spot is made up of a plurality of unique oligonucleotides that are capable of hybridizing to different regions of a corresponding target nucleic acid. A plurality of different target nucleic acids are represented on the arrays, where a particular target nucleic acid may correspond to only one or a plurality of different oligonucleotide probe spots on the array. In a preferred embodiment, all of the target nucleic acids represented on the array are of the same type. The subject arrays find particular use in gene expression assays. In further describing the subject invention, the arrays first will be described in general terms. Next, methods for their preparation are described. Following this, a review of representative applications in which the subject arrays may be employed is provided. Finally, a description of representative specific array types falling within the scope of the invention will be provided.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Arrays of the Subject Invention-General Description

Array Structure

The arrays of the subject invention have a plurality of probe oligonucleotide spots stably associated with a surface of a solid support. Each oligonucleotide spot on the array comprises an oligonucleotide probe composition of known identity, usually of known sequence, as described in greater detail below. The oligonucleotide spots on the array may be any convenient shape, but will typically be circular, elliptoid, oval or some other analogously curved shape. The density of the spots on the solid surface is at least about 5/cm² and usually at least about 10/cm² but does not exceed about 1000/cm², and usually does not exceed about 500/cm² or 400/cm², and more usually does not exceed about 300/cm². The spots may be arranged in a spatially defined and physically addressable manner, in any convenient pattern across or over the surface of the array, such as in rows and columns so as to form a grid, in a circular pattern, and the like, where generally the pattern of spots will be present in the form of a grid across the surface of the solid support.

In the subject arrays, the spots of the pattern are stably associated with the surface of a solid support, where the support may be a flexible or rigid support. By “stably associated” it is meant that the oligonucleotides of the spots maintain their position relative to the solid support under hybridization and washing conditions. As such, the oligonucleotide members which make up the spots can be non-covalently or covalently stably associated with the support surface based on technologies well know to those of skill in the art. Examples of non-covalent association include non-specific adsorption, binding based on electrostatic (e.g. ion, ion pair interactions), hydrophobic interactions, hydrogen bonding interactions, specific binding through a specific binding pair member covalently attached to the support surface, and the like. Examples of covalent binding include covalent bonds formed between the spot oligonucleotides and a functional group present on the surface of the rigid support, e.g.—OH, where the functional group may be naturally occurring or present as a member of an introduced linking group, as described in greater detail below.

As mentioned above, the array is present on either a flexible or rigid substrate. By flexible is meant that the support is capable of being bent, folded or similarly manipulated without breakage. Examples of solid materials which are flexible solid supports with respect to the present invention include membranes, flexible plastic films, and the like. By rigid is meant that the support is solid and does not readily bend, i.e. the support is not flexible. As such, the rigid substrates of the subject arrays are sufficient to provide physical support and structure to the polymeric targets present thereon under the assay conditions in which the array is employed, particularly under high throughput handling conditions. Furthermore, when the rigid supports of the subject invention are bent, they are prone to breakage.

The solid supports upon which the subject patterns of spots are presented in the subject arrays may take a variety of configurations ranging from simple to complex, depending on the intended use of the array. Thus, the substrate could have an overall slide or plate configuration, such as a rectangular or disc configuration. In many embodiments, the substrate will have a rectangular cross-sectional shape, having a length of from about 10 mm to 200 mm, usually from about 40 to 150 mm and more usually from about 75 to 125 mm and a width of from about 10 mm to 200 mm, usually from about 20 mm to 120 mm and more usually from about 25 to 80 mm, and a thickness of from about 0.01 mm to 5.0 mm, usually from about 0.1 mm to 2 mm and more usually from about 0.2 to 1 mm. Thus, in one embodiment the support may have a micro-titre plate format, having dimensions of approximately 125×85 mm.

The substrates of the subject arrays may be fabricated from a variety of materials. The materials from which the substrate is fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, is of particular interest in this embodiment. For rigid substrates, specific materials of interest include: glass; plastics, e.g. polytetrafluoroethylene, polypropylene, polystyrene, polycarbonate, and blends thereof, and the like; metals, e.g. gold, platinum, and the like; etc.

The substrates of the subject arrays comprise at least one surface on which the pattern of spots is present, where the surface may be smooth or substantially planar, or have irregularities, such as depressions or elevations. The surface on which the pattern of spots is present may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polynreas, polyamides, polyethyleneamines, polyarylene sulfides, polysiloxanes, polyimides, polyacetates, polyacrylamides, and the like, where the polymers may be hetero- or homopolymeric, and may or may not have separate functional moieties attached thereto, e.g. conjugated.

The total number of spots on the substrate will vary depending on the number of different oligonucleotide spots (oligonucleotide probe compositions) one wishes to display on the surface, as well as the number of control spots, orientation spots, calibrating spots and the like, as may be desired depending on the particular application in which the subject arrays are to be employed. Generally, the pattern present on the surface of the array will comprise at least about 10 distinct oligonucleotide spots, usually at least about 20 distinct oligonucleotide spots, and more usually at least about 50 distinct oligonucleotide spots, where the number of oligonucleotide spots may be as high as 10,000 or higher, but will usually not exceed about 5,000 distinct oligonucleotide spots, and more usually will not exceed about 3,000 distinct oligonucleotide spots and in many instances will not exceed about 1,000. In many embodiments, it is preferable to have each distinct oligonucleotide spot or probe composition presented in duplicate, i.e. so that there are two spots for each distinct oligonucleotide probe composition of the array. In certain embodiments, the number of spots will range from about 200 to 600.

In the arrays of the subject invention (particularly those designed for use in high throughput applications, such as high throughput analysis applications), a single pattern of oligonucleotide spots may be present on the array or the array may comprise a plurality of different oligonucleotide spot patterns, each pattern being as defined above. When a plurality of different oligonucleotide spot patterns are present, the patterns may be identical to each other, such that the array comprises two or more identical oligonucleotide spot patterns on its surface, or the oligonucleotide spot patterns may be different, e.g. in arrays that have two or more different types of target nucleic acids represented on their surface, e.g an array that has a pattern of spots corresponding to human genes and a pattern of spots corresponding to mouse genes. Where a plurality of spot patterns are present on the array, the number of different spot patterns is at least 2, usually at least 6, more usually at least 24 or 96, where the number of different patterns will generally not exceed about 384.

Where the array comprises a plurality of oligonucleotide spot patterns on its surface, preferably the array comprises a plurality of reaction chambers, wherein each chamber has a bottom surface having associated therewith an pattern of oligonucleotide spots and at least one wall, usually a plurality of walls surrounding the bottom surface. Such array configurations and the preparation thereof is further described in U.S. patent application Ser. No. 08/974,298 filed on Nov. 19, 1997, the disclosure of which is herein incorporated by reference. Of particular interest in many embodiments are arrays in which the same pattern of spots in reproduced in 24 or 96 different reaction chambers across the surface of the array.

Within any given pattern of spots on the array, there may be a single spot that corresponds to a given target or a number of different spots that correspond to the same target, where when a plurality of different spots are present that correspond to the same target, the probe compositions of each spot that corresponds to the same target may be identical of different. In other words, a plurality of different targets are represented in the pattern of spots, where each target may correspond to a single spot or a plurality of spots, where the oligonucleotide probe composition among the plurality of spots corresponding to the same target may be the same or different. Where a plurality of spots (of the same or different composition) corresponding to the same target is present on the array, the number of spots in this plurality will be at least about 2 and may be as high as 10, but will usually not exceed about 5. The number of different targets represented on the array is at least about 2, usually at least about 10 and more usually at least about 20, where in many embodiments the number of different targets, e.g. genes, represented on the array is at least about 50. The number of different targets represented on the array may be as high as 1000 or higher, but will usually not exceed about 800 and more usually will not exceed about 700. A target is considered to be represented on an array if it is able to hybridize to one or more probe compositions on the array.

The total amount or mass of oligonucleotides present in each spot will be sufficient to provide for adequate hybridization and detection of target nucleic acid during the assay in which the array is employed. Generally, the total mass of oligonucleotides in each spot will be at least about 0.1 ng, usually at least about 0.5 ng and more usually at least about 1 ng, where the total mass may be as high as 1000 ng or higher, but will usually not exceed about 20 ng and more usually will not exceed about 10 ng. The copy number of all of the oligonucleotides in a spot will be sufficient to provide enough hybridization sites for target molecule to yield a detectable signal, and will generally range from about 0.01 fmol to 50 fmol, usually from about 0.05 fmol to 20 fmol and more usually from about 0.1 fmol to 5 fmol. The molar ratio or copy number ratio of different oligonucleotides within each spot may be about equal or may be different, wherein when the ratio of unique oligonucleotides within each spot differs, the magnitude of the difference will usually be at least 2 to 10 fold but will generally not exceed about 100 fold. Where the spot has an overall circular dimension, the diameter of the spot will generally range from about 10 to 5,000 μm, usually from about 20 to 1,000 μm and more usually from about 50 to 500 μm. The surface area of each spot is at least about 100 μm², usually at least about 400 μm² and more usually at least about 800 μm², and may be as great as 25 mm² or greater, but will generally not exceed about 5 mm², and usually will not exceed about 1 mm².

In a preferred embodiment of the invention, each of the oligonucleotide spots in the array comprising the oligonucleotide probe compositions correspond to the same kind of gene; i.e. genes that all share some common characteristic or can be grouped together based on some common feature, such as species of origin, tissue or cell of origin, functional role, disease association, etc. In this embodiment, each of the different target nucleic acids that correspond to the different probe spots on the array are of the same type, i.e. that are coding sequences of the same type of gene. As such, the arrays of this embodiment of the subject invention will be of a specific array type, where representative array types include: human arrays, cancer arrays, apoptosis arrays, neuroarrays, mouse arrays, arrays of human stress genes, arrays of oncogenes and tumor suppressors, arrays of signal transduction genes, and the like, where some of these representative arrays are described in greater detail below.

With respect to the oligonucleotide probes that correspond to a particular type or kind of gene, type or kind can refer to a plurality of different characterizing features, where such features include: species specific genes, where specific species of interest include eukaryotic species, such as mice, rats, rabbits, pigs, primates, humans, etc.; function specific genes, where such genes include oncogenes, apoptosis genes, cytokines, receptors, protein kinases, etc.; genes specific for or involved in a particular biological process, such as apoptosis, differentiation, stress response, aging, proliferation, etc.; cellular mechanism genes, e.g. cell-cycle, signal transduction, metabolism of toxic compounds, etc.; disease associated genes, e.g. genes involved in cancer, schizophrenia, diabetes, high blood pressure, atherosclerosis, viral-host interaction and infection diseases, etc.; location specific genes, where locations include organ, such as heart, liver, prostate, lung etc., tissue, such as nerve, muscle, connective, etc., cellular, such as axonal, lymphocytic, etc, or subcellular locations, e.g. nucleus, endoplasmic reticulum, Golgi complex, endosome, lysosome, peroxisome, mitochondria, cytoplasm, cytoskeleton, plasma membrane, extracellular space, chromosome-specific genes; specific genes that change expression level over time, e.g. genes that are expressed at different levels during the progression of a disease condition, such as prostate genes which are induced or repressed during the progression of prostate cancer.

In addition to the oligonucleotide spots comprising the oligonucleotide probe compositions (i.e. oligonucleotide probe spots), the subject arrays may comprise one or more additional spots of polynucleotides which do not correspond to target nucleic acids as defined above, such as target nucleic acids of the type or kind of gene represented on the array in those embodiments in which the array is of a specific type. In other words, the array may comprise one or more spots that are made of non “unique” oligonucleotides or polynucleotides, i.e common oligonucleotides or polynucleotides. For example, spots comprising genomic DNA may be provided in the array, where such spots may serve as orientation marks. Spots comprising plasmid and bacteriophage genes, genes from the same or another species which are not expressed and do not cross hybridize with the cDNA target, and the like, may be present and serve as negative controls. In addition, spots comprising a plurality of oligonucleotides complimentary to housekeeping genes and other control genes from the same or another species may be present, which spots serve in the normalization of mRNA abundance and standardization of hybridization signal intensity in the sample assayed with the array. Orientation spots may also be included on the array, where such spots serve to simplify image analysis of hybrid patterns. These latter types of spots are distinguished from the oligonucleotide probe spots, i.e. they are non-probe spots.

The array may further comprise mismatch control probes. Mismatch controls may be provided for the probes to the target genes, for expression level controls or for normalization controls. Mismatch controls are oligonucleotide probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g. stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross-hybridization to a nucleic acid in the sample other than the target to which the probe is directed. Mismatch probes thus indicate whether a hybridization is specific or not. For example, if the target is present the perfect match probes should be consistently brighter than the mismatch probes. In addition, if all central mismatches are present, the mismatch probes can be used to detect a mutation. Finally, the difference in intensity between the perfect match and the mismatch probe (I(PM)-I(MM)) provides a good measure of the concentration of the hybridized material.

Oligonucleotide Probes of the Arrays

Each oligonucleotide spot on the surface of the substrate is made up of a unique oligonucleotide probe composition. By “oligonucleotide probe composition” is meant a collection, population, or plurality of unique oligonucleotides. Each of the oligonucleotides present in the oligonucleotide probe composition is capable of hybridizing to a distinct or different region of the same target nucleic acid to which they correspond, i.e. the target nucleic acid corresponding to the spot in which the oligonucleotide composition is positioned. By “capable of hybridizing to distinct or different regions” is meant that each unique oligonucleotide in the probe composition hybridizes to a different stretch of nucleotide residues in the target nucleic acid, where the different stretches or regions of the target nucleic acid may be continuous, separated by one or more nucleotide residues, or overlapping but physically belong to the same target molecule.

With respect to probe compositions that do not correspond to the same target, the unique oligonucleotides are chosen so that each distinct unique oligonucleotide is not homologous with any other distinct unique oligonucleotide. In other words, each distinct oligonucleotide of a probe composition corresponding a first target does not cross-hybridize with, or have the same sequence as, any other distinct unique oligonucleotide on of any probe composition corresponding to a different target, i.e. an oligonucleotide of any other oligonucleotide probe composition that is represented on the array. As such, the sense or anti-sense nucleotide sequence of each unique oligonucleotide of a probe composition will have less than 90% homology, usually less than 85% homology, and more usually less than 80% homology with any other different oligonucleotide of a probe composition corresponding to a different target of the array, where homology is determined by sequence analysis comparison using the FASTA program using default settings. The sequence of unique oligonucleotides in the probe compositions are not conserved sequences found in a number of different genes (at least two), where a conserved sequence is defined as a stretch of from about 15 to 150 nucleotides which have at least about 90% sequence identity, where sequence identity is measured as above. Again, the length of the oligonucleotide will be shorter than the mRNA to which it corresponds. However, where more than one probe composition of the array corresponds to the same target, the same unique oligonucleotide may be present in two or more of these probe compositions that all correspond to the same target. In other words, among such probe compositions that correspond to the same target, such probe compositions may have one or more unique oligonucleotides in common.

The unique oligonucleotides of the subject probe compositions will generally have a length of from about 15 to 150 nt, usually from 25 to 100 nt, and more usually 30 to 70 nt. The number of different unique oligonucleotides in each probe composition will range from about 2 to 50 or 3 to 50, usually from about 3 to 20, and more usually from about 3 to 10.

Within each spot, all of the different oligonucleotides probes should have substantially the same melting temperature to the target. In other words, the melting temperature or Tₘ of any double stranded complex formed between any one oligonucleotide and the target should not be substantially different from the Tₘ of any other double stranded complex formed between the target and any other oligonucleotide of the same probe composition. By “substantially the same” is meant that any difference in Tₘ will not exceed more than 30° C., usually not more than about 20° C. and more usually not more than about 10° C.

The oligonucleotides of each probe composition are further characterized by having a GC content of from about 35% to 80%. The oligonucleotides are also characterized by the substantial absence of secondary structures and long homopolymeric stretches, e.g. polyA stretches, such that in any give homopolymeric stretch, the number of contiguous identical nucleotide bases does not exceed 5.

The oligonucleotide probe compositions are yet further characterized in that the individual oligonucleotides of each probe bind to the target in a cooperative fashion, i.e. they cooperatively hybridize to the target. By cooperative fashion is meant that the probe compositions of the subject invention acheive at least one of: (a) ds complexes with higher Tₘ values; (b) increased retention or hybridization efficiency as compared to single probes; and (d) increased hybridization rate as compared to singled probes. Thus, by cooperative fashion or manner is meant that, in certain embodiments, the probes help each other to bind to the target to produce a double-stranded complex that has a higher melting temperature or Tₘ than the Tₘ of any double-stranded complex of the target and a single oligonucleotide of the probe composition. For example, if an oligonucleotide probe is made up of three different oligonucleotides, (1, 2 & 3) then the double-stranded complex formed by the target and all three oligonucleotides has a Tₘ that exceeds the double-stranded complex produced between the target and oligonucleotide 1, oligonucleotide 2 or oligonucleotide 3. The magnitude of the difference in Tₘ is generally at least about 3° C., and preferably at least about 5° C. and more preferably at least about 10° C. Alternatively or additionally, the cooperative probes of the subject probe compositions can have an increased hybridization rate to the target, as compared to a single probe, where the increase in hybridization rate will typically be at least about 2-fold, and more often at least about 5-fold. In addition or alternatively, the subject cooperative probes may result in increased retention or hybridization efficiency as compared to single probes, where the increase will typically be at least about 2-fold and more often at least about 5-fold.

Depending on the nature of the target nucleic acid, all of the oligonucleotides within a given probe composition may bind to the same nucleic acid strand or to different nucleic strands. Thus, where the target is single stranded, i.e. MRNA or cDNA, the oligonucleotides will bind to the same target strand. In contrast, where the target is double stranded, such as ds cDNA, the oligonucleotides may bind to the same strand or to different strands, e.g. one oligonucleotide may bind to the sense strand and one may bind to the anti-sense strand.

Within a given probe composition, the various oligonucleotides may or may not interact with each other in binding to the target. Where the oligonucleotides do not interact with each other when binding to the target, each oligonucleotide will bind separately to the target without interacting, e.g. binding, to any other oligonucleotide in the probe composition. In contrast, where the oligonucleotides interact with each other in binding to the target, the oligonucleotides may have regions of partial complementarity to each other and/or have other stable association means with each other, such as specific binding pairs, etc., which provide for the desired interaction of the various oligonucleotides of the probe composition.

The oligonucleotide probe compositions that make up each oligonucleotide spot on the array will be substantially, usually completely, free of non-nucleic acids, i.e. the probe compositions will not comprise non-nucleic acid biomolecules found in cells, such as proteins, lipids, and polysaccharides. In other words, the oligonucleotide spots of the arrays are substantially, if not entirely, free of non-nucleic acid cellular constituents.

The oligonucleotide probes may be nucleic acid, e.g. RNA, DNA, or nucleic acid mimetics, e.g. such as nucleic acids comprising non-naturally occurring heterocyclic nitrogeneous bases, peptide-nucleic acids, locked nucleic acids (see Singh & Wengel, Chem. Commun. (1998) 1247-1248); and the like.

Array Preparation

The subject arrays can be prepared using any convenient means. One means of preparing the subject arrays is to first synthesize the oligonucleotides for each spot and then deposit the oligonucleotides as a spot on the support surface. The oligonucleotides may be prepared using any convenient methodology, such as automated solid phase synthesis protocols, and like, where such techniques are well known to those of skill in the art.

In determining the specific oligonucleotides of the probe compositions, the oligonucleotide should be chosen so that is capable of hybridizing to a region of the target nucleic acid or gene having a sequence unique to that gene. Different methods may be employed to choose the specific region of the gene to which the oligonucleotide probe is to hybridize. Thus, one can use a random approach based on availability of a gene of interest. However, instead of using a random approach which is based on availability of a gene of interest, a rational design approach may also be employed to choose the optimal sequence for the hybridization array. Preferably, the region of the gene that is selected in preparing the oligonucleotide probe is chosen based on the following criteria. First, the sequence that is chosen should yield an oligonucleotide probe that does not cross-hybridize with, or is homologous to, any other oligonucleotide probe for other spots present on the array that do not corresponding to the target gene. Second, the sequence should be chosen such that the oligonucleotide probe has a low homology to a nucleotide sequence found in any other gene, whether or not the gene is to be represented on the array from the same species of origin, e.g. for a human array, the sequence will not be homologous to any other human genes. As such, sequences that are avoided include those found in: highly expressed gene products, structural RNAS, repeated sequences found in the sample to be tested with the array and sequences found in vectors. A further consideration is to select sequences which provide for minimal or no secondary structure, structure which allows for optimal hybridization but low non-specific binding, equal or similar thermal stabilities, and optimal hybridization characteristics.

The prepared oligonucleotides may be spotted on the support using any convenient methodology, including manual techniques, e.g. by micro pipette, ink jet, pins, etc., and automated protocols, where the different oligonucleotides of each spot can be mixed together as described above and spotted or spotted separately in the same spot location in a sequential fashion. Of particular interest is the use of an automated spotting device, such as the Beckman Biomek 2000 (Beckman Instruments).

Methods of Using the Subject Arrays

The subject arrays find use in a variety of different applications in which one is interested in detecting the occurrence of one or more binding events between target nucleic acids and probes on the array and then relating the occurrence of the binding event(s) to the presence of a target(s) in a sample. In general, the device will be contacted with the sample suspected of containing the target under conditions sufficient for binding of any target present in the sample to complementary oligonucleotides present on the array. Generally, the sample will be a fluid sample and contact will be achieved by introduction of an appropriate volume of the fluid sample onto the array surface, where introduction can be through delivery ports, direct contact, deposition, and the like.

Generation of Labeled Target

Targets may be generated by methods known in the art. mRNA can be labeled and used directly as a target, or converted to a labeled cDNA target. Usually, MRNA is labeled directly using chemically, photochemically or enzymatically activated labeling compounds, such as photobiotin (Clontech, Palo Alto, Calif.), Dig-Chem-Link (Boehringer), and the like. Generally, methods for generating labeled cDNA probes include the use of oligonucleotide primers. Primers that may be employed include oligo dT, random primers, e.g. random hexamers and gene specific primers, as described in PCT/US98/10561, the disclosure of which is herein incorporated by reference. Where gene specific primers are employed, the gene specific primers are preferably those primers that correspond to the different oligonucleotide spots on the array. Thus, one will preferably employ gene specific primers for each different oligonucleotide that is present on the array, so that if the gene is expressed in the particular cell or tissue being analyzed, labeled target will be generated from the sample for that gene. In this manner, if a particular gene present on the array is expressed in a particular sample, the appropriate target will be generated and subsequently identified. For each target represented on the array, a single gene specific primer may be employed or a plurality of different gene specific primers may be employed, where when a plurality are used to produce the target, the number will generally not exceed about 5. Generally, in preparing the target from template nucleic acid, e.g. mRNA, the gene specific primers will hybridize to a region of the template that is downstream from the region to which the probes are homologous, e.g. to which the probes are complementary or have the same sequence. However, in certain embodiments the gene specific primers may be complementary to the oligonucleotide probes. The cDNA probe can be further amplified by PCR or can be converted (linearly amplified) using phage coded RNA polymerase transcriptionn of dsDNA. See PCT/US98/1056, the disclosure of which is herein incorporated by reference.

A variety of different protocols may be used to generate the labeled target nucleic acids, as is known in the art, where such methods typically rely in the enzymatic generation of the labeled target using the initial primer. Labeled primers can be employed to generate the labeled target. Alternatively, label can be incorporated during first strand synthesis or subsequent synthesis, labeling or amplification steps in order to produce labeled target. Representative methods of producing labeled target are disclosed in PCT/US98/10561, the disclosure of which is herein incorporated by reference.

Hybridization and Detection

As mentioned above, following preparation of the target nucleic acid from the tissue or cell of interest, the target nucleic acid is then contacted with the array under hybridization conditions, where such conditions can be adjusted, as desired, to provide for an optimum level of specificity in view of the particular assay being performed. Suitable hybridization conditions are well known to those of skill in the art and reviewed in Maniatis et al, supra and WO 95/21944. In analyzing the differences in the population of labeled target nucleic acids generated from two or more physiological sources using the arrays described above, each population of labeled target nucleic acids are separately contacted to identical probe arrays or together to the same array under conditions of hybridization, preferably under stringent hybridization conditions, such that labeled target nucleic acids hybridize to complementary probes on the substrate surface.

Where all of the target sequences comprise the same label, different arrays will be employed for each physiological source (where different could include using the same array at different times). Alternatively, where the labels of the targets are different and distinguishable for each of the different physiological sources being assayed, the opportunity arises to use the same array at the same time for each of the different target populations. Examples of distinguishable labels are well known in the art and include: two or more different emission wavelength fluorescent dyes, like Cy3 and Cy5, two or more isotopes with different energy of emission, like ³²p and ³³p, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Following hybridization, non-hybridized labeled nucleic acid is removed from the support surface, conveniently by washing, generating a pattern of hybridized nucleic acid on the substrate surface. A variety of wash solutions are known to those of skill in the art and may be used.

The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, colorimetric measurement, light emission measurement, light scattering, and the like.

Following detection or visualization, the hybridization patterns may be compared to identify differences between the patterns. Where arrays in which each of the different probes corresponds to a known gene are employed, any discrepancies can be related to a differential expression of a particular gene in the physiological sources being compared.

The provision of appropriate controls on the arrays permits a more detailed analysis that controls for variations in hybridization conditions, cell health, non-specific binding and the like. Thus, for example, in a preferred embodiment, the hybridization array is provided with normalization controls as described supra. These normalization controls are probes complementary to control sequences added in a known concentration to the sample. Where the overall hybridization conditions are poor, the normalization controls will show a smaller signal reflecting reduced hybridization. Conversely, where hybridization conditions are good, the normalization controls will provide a higher signal reflecting the improved hybridization. Normalization of the signal derived from other probes in the array to the normalization controls thus provides a control for variations in hybridization conditions. Typically, normalization is accomplished by dividing the measured signal from the other probes in the array by the average signal produced by the normalization controls. Normalization may also include correction for variations due to sample preparation and amplification. Such normalization may be accomplished by dividing the measured signal by the average signal from the sample preparation/amplification control probes. The resulting values may be multiplied by a constant value to scale the results.

As indicated above, the subject arrays can include mismatch controls. In a preferred embodiment, there is a mismatch control having a central mismatch for every probe (except the normalization controls) in the array. It is expected that after washing in stringent conditions, where a perfect match would be expected to hybridize to the probe, but not to the mismatch, the signal from the mismatch controls should only reflect non-specific binding or the presence in the sample of a nucleic acid that hybridizes with the mismatch. Where both the probe in question and its corresponding mismatch control both show high signals, or the mismatch shows a higher signal than its corresponding test probe, there is a problem with the hybridization and the signal from those probes is ignored. The difference in hybridization signal intensity between the target specific probe and its corresponding mismatch control is a measure of the discrimination of the target-specific probe. Thus, in a preferred embodiment, the signal of the mismatch probe is subtracted from the signal from its corresponding test probe to provide a measure of the signal due to specific binding of the test probe.

The concentration of a particular sequence can then be determined by measuring the signal intensity of each of the probes that bind specifically to that gene and normalizing to the normalization controls. Where the signal from the probes is greater than the mismatch, the mismatch is subtracted. Where the mismatch intensity is equal to or greater than its corresponding test probe, the signal is ignored. The expression level of a particular gene can then be scored by the number of positive signals (either absolute or above a threshold value), the intensity of the positive signals (either absolute or above a selected threshold value), or a combination of both metrics (e.g., a weighted average).

In certain embodiments, normalization controls are often unnecessary for useful quantification of a hybridization signal. Thus, where optimal probes have been identified, the average hybridization signal produced by the selected optimal probes provides a good quantified measure of the concentration of hybridized nucleic acid.

Where mismatch controls are present, the detecting step may comprise calculating the difference in hybridization signal intensity between each of the oligonucleotide probes and its corresponding mismatch control probe. The detection step may further comprise calculating the average difference in hybridization signal intensity between each of the oligonucleotide probes and its corresponding mismatch control probe for each gene.

Utility

The subject methods find use in, among other applications, differential gene expression assays. Thus, one may use the subject methods in the differential expression analysis of: (a) diseased and normal tissue, e.g. neoplastic and normal tissue, (b) different tissue or tissue types; (c) developmental stage; (d) response to external or internal stimulus; (e) response to treatment; and the like. The subject arrays therefore find use in broad scale expression screening for drug discovery, diagnostics and research, as well as studying the effect of a particular active agent on the expression pattern of genes in a particular cell, where such information can be used to reveal drug toxicity, carcinogenicity, etc., environmental monitoring, disease research and the like.

Kits

Also provided are kits for performing analyte binding assays using the subject devices, where kits for carrying out differential gene expression analysis assays are preferred. Such kits according to the subject invention will at least comprise the subject arrays. The kits may further comprise one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

Specific Array Types of the Subject Invention

As mentioned above, in certain preferred embodiments, the subject array is of a specific type in that all of the target nucleic acid represented on the array by the oligonucleotide probe compositions are the same type of target nucleic acid, i.e. they are the same type of gene. A variety of specific array types are provided by the subject invention. Specific array types of interest include those described earlier, including: human, cancer, apoptosis, mouse, human stress, oncogene and tumor suppressor, cell-cell interaction, cytokine and cytokine receptor, rat, rat stress, blood, mouse stress, neuroarray, and the like. For a more detailed description of the different target nucleic acids represented on each of these types of arrays, see PCT/US98/10561 the disclosure of which is herein incorporated by reference.

It is evident from the above discussion that the subject arrays provide for a significant advance in the field. With the subject arrays, using a plurality of unique oligonucleotides instead of a single oligonucleotide allows one to attain high specificity of hybridization with a minimum of non-specific binding, where these attributes result from the effects of cooperative interaction of the plurality of oligonucleotides with the target. As such, the subject arrays combine the robustness of cDNA arrays—high melting temperature of target/probe complexes, high efficiency of target retention (binding) and high sensitivity—with the high resolution power of oligonucleotide arrays—ability to distinguish highly homologous sequences which differ only by 100-200 nucleotides. In addition, the subject arrays are no more expensive or difficult to produce than standard oligonucleotide arrays, and as such are particularly suited for high throughput expression analysis and diagnostic applications. Furthermore, the subject arrays should be less expensive to produce than cDNA arrays, and provide more reproducible results providing for improved compliance with governmental regulations regarding diagnostic assays. Assays conducted with the subject arrays yield a large amount of information regarding the expression of numerous different and important genes in a particular sample at substantially the same time, and thus have use in many different types of applications, including drug discovery and characterization, disease research, and the like.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. An array comprising at least one pattern of probe oligonucleotide spots stably associated with the surface of a solid support, wherein each probe oligonucleotide spot corresponds to a target nucleic acid and comprises an oligonucleotide probe composition made up of a plurality of unique oligonucleotides.

2. The array according to claim 1, wherein said plurality of unique oligonucleotides are capable of hybridizing to different regions of the corresponding target nucleic acid of the oligonucleotide spot in which they are positioned.

3. The array according to claim 2, wherein said plurality of unique oligonucleotides hybridize to non-overlapping regions of said target nucleic acid.

4. The array according to claim 2, wherein said plurality of unique oligonucleotides hybridize to overlapping regions of said target nucleic acid.

5. The array according to claim 1, wherein two or more different target nucleic acids are represented in said pattern.

6. The array according to claim 5, wherein each probe oligonucleotide spot in said pattern corresponds to a different target nucleic acid.

7. The array according to claim 5, wherein two or more probe oligonucleotide spots in said pattern correspond to the same target nucleic acid.

8. The array according to claim 1, wherein said array comprises a plurality of said patterns.

9. The array according to claim 8, wherein said plurality of patterns are separated from each other by walls.

10. The array according to claim 1, wherein the length of each of said oligonucleotides ranges from about 15 to 150 nucleotides.

11. The array according to claim 1, wherein said array further comprises at least one mismatch probe.

12. The array according to claim 1, wherein the number of oligonucleotides of each of said oligonucleotide probe compositions ranges from about 3 to 50.

13. The array according to claim 1, wherein all of said oligonucleotide spots correspond to the same type of target nucleic acid.

14. The array according to claim 1, wherein the density of spots on said array does not exceed about 1000/cm².

15. The array according to claim 14, wherein the density of spots on said array does not exceed about 400/cm².

16. The array according to claim 1, wherein the number of spots on said array ranges from about 50 to 10,000.

17. The array according to claim 1, wherein the number of spots on said array ranges from about 50 to 1,000.

18. An array comprising a pattern of probe oligonucleotide spots stably associated with the surface of a solid support, wherein each probe oligonucleotide spot corresponds to a target nucleic acid and comprises an oligonucleotide probe composition made up of 3 to 50 unique oligonucleotides of from about 15 to 150 nucleotides in length, wherein each unique oligonucleotide is capable of hybridizing to a different region of the corresponding target nucleic acid of the probe oligonucleotide spot in which it is positioned.

19. The array according to claim 18, wherein said plurality of unique oligonucleotides hybridize to non-overlapping regions of said target nucleic acid.

20. The array according to claim 18, wherein said plurality of unique oligonucleotides hybridize to overlapping regions of said target nucleic acid.

21. The array according to claim 18, wherein said unique oligonucleotides of each spot cooperatively hybridize to said target.

22. The array according to claim 18, wherein ten or more different target nucleic acids are represented in said pattern.

23. The array according to claim 22, wherein each probe oligonucleotide spot in said pattern corresponds to a different target nucleic acid.

24. The array according to claim 22, wherein two or more probe oligonucleotide spots in said pattern correspond to the same target nucleic acid.

25. The array according to claim 18, wherein the length of each of said unique oligonucleotides ranges from about 25 to 100 nucleotides.

26. The array according to claim 18, wherein the number of unique oligonucleotides of each of said oligonucleotide probe compositions ranges from about 3 to 20.

27. The array according to claim 18, wherein the density of spots on said array does not exceed about 1000/cm².

28. The array according to claim 18, wherein the density of spots on said array does not exceed about 400/cm².

29. The array according to claim 18, wherein the number of spots on said array ranges from about 50 to 10,000.

30. The array according to claim 18, wherein the number of spots on said array ranges from about 50 to 1,000.

31. An array comprising a pattern of probe oligonucleotide spots of a density that does not exceed about 400 spots/cm² stably associated with the surface of a solid support, wherein each probe oligonucleotide spot corresponds to a different target nucleic acid and comprises an oligonucleotide probe composition made up of 3 to 20 unique oligonucleotides of from about 25 to 100 nucleotides in length, wherein each unique oligonucleotide is capable of hybridizing to a different region of the corresponding target nucleic acid of the probe oligonucleotide spot in which it is positioned.

32. The array according to claim 31, wherein said unique oligonucleotides hybridize to non-overlapping regions of said target nucleic acid.

33. The array according to claim 31, wherein said unique oligonucleotides hybridize to overlapping regions of said target nucleic acid.

34. The array according to claim 31, wherein said array comprises a plurality of said patterns.

35. The array according to claim 34, wherein said plurality of patterns are separated from each other by walls.

36. The array according to claim 31, wherein the number of spots on said array ranges from about 50 to 10,000.

37. The array according to claim 31, wherein the number of spots on said array ranges from about 50 to 1,000.

38. A method of preparing an array comprising at least one pattern of probe oligonucleotide spots stably associated with the surface of a solid support, wherein each probe oligonucleotide spot corresponds to a target nucleic acid and comprises an oligonucleotide probe composition made up of a plurality of unique oligonucleotides, said method comprising:
generating said unique oligonucleotides; and
stably associating said unique oligonucleotides on the surface of said solid support in a manner sufficient to produce said array.

39. The method according to claim 38, wherein said solid support is flexible.

40. The method according to claim 39, wherein said solid support is a nylon.

41. The method according to claim 38, wherein said solid support is rigid.

42. The method according to claim 41, wherein said solid support is glass.

43. The method according to claim 38, wherein said method further comprises the step of selecting said unique oligonucleotides.

44. The method according to claim 43, wherein said unique oligonucleotides are not homologous to any other unique oligonucleotide of any other oligonucleotide probe composition corresponding to a different target nucleic acid.

45. The array produced according to the method of claim 38.

46. A hybridization assay comprising the steps of:
contacting at least one labeled target nucleic acid sample with an array according to claim 1 under conditions sufficient to produce a hybridization pattern; and
detecting said hybridization pattern.

47. The method according to claim 46, wherein said method further comprises washing said array prior to said detecting step.

48. The method according to claim 46, wherein said method further comprises preparing said labeled target nucleic acid sample.

49. The method according to claim 48, wherein said preparing comprises conjugating a detectable label to a functionalized target nucleic acid.

50. The method according to claim 46, where said method further comprises:
generating a second hybridization pattern; and
comparing said hybridization patterns.

51. The method according to claim 50, wherein said hybridization patterns are generated on the same array.

52. The method according to claim 50, wherein the second hybridization patters are generated on different arrays.

53. A kit for use in a hybridization assay, said kit comprising:
an array according to claim 1.

54. The kit according to claim 53, wherein said kit further comprises reagents for generating a labeled target nucleic acid sample.

55. The kit according to claim 53, wherein said kit further comprises a hybridization buffer.

56. The kit according to claim 53, wherein said kit further comprises a wash medium.
